(19) United States
(12) Patent Application Publication
(10) Pub. No.: US 2010/0003696 A1
(43) Pub. Date: Jan. 7, 2010

(54) METHOD OF DIAGNOSING AND MONITORING THE PROGRESS OF MULTIPLE SCLEROSIS, AND THE USE OF A TEST KIT THEREFOR

(76) Inventor: Torsten Matthias, Wendelsheim (DE)

Correspondence Address: BUCHANAN INGERSOLL & ROONEY PC, P.O. BOX 1404, ALEXANDRIA, VA, 22313-1404, US

(21) Appl. No.: 12/441,175

(22) PCT Filed: Sep. 14, 2007
(86) PCT No.: PCT/DE07/01664

§ 371 Date: May 14, 2009

(30) Foreign Application Priority Data
Sep. 15, 2006 (DE) 10 2006 043 369.6

(51) Int. Cl. G01N 33/53

(52) U.S. Cl. 435/7.1

(57) ABSTRACT

There is a described method and a test kit for diagnosing and monitoring the progress of multiple sclerosis by determining autoantibodies in bodily fluids by determining those antibodies which bind to a spectrin. A typical spectrin is alpha-fodrin.

### CROSS-REFERENCE TO RELATED APPLICATIONS

This application claims the benefit of co-pending International Patent Application No. PCT/DE2007/001664 entitled “Method of Diagnosing and Monitoring the Progress of Multiple Sclerosis, and the Use of a Test Kit Therefore,” filed on Sep. 14, 2007 and German Patent Application No. DE 10 2006 043 369.6, filed Sep. 15, 2006. Both of those applications are incorporated by reference herein.

### BACKGROUND OF THE INVENTION

### Field of the Invention

The invention relates to a method for the diagnosis of multiple sclerosis, in particular for the diagnosis and monitoring of the progression of imminent or incipient inflammatory relapses as well as the use of a test kit therefore.

Multiple sclerosis (MS), which is also called encephalomyelitis disseminata, is a primary inflammatory disease of the central nervous system. However, it does not represent a homogeneous form of the disease but instead describes a group of inflammatory demyelinating disorders of the central nervous system with very variable clinical courses. At the same time, the clinical course can range from a single event or a benign course of the disease with only a few relapses and minimal risk of disability to the classic relapsing-remitting forms with or without secondary, chronic progressive clinical forms.

The onset is frequently between 20 and 50 years of age, with women being affected twice as often as men. An association with HLA antigens, particularly HLA-DR2 as well as HLA-DW2, also occurs. Multiple sclerosis is an autoimmune disease, in which the body's own immune defenses attack and destroy the Schwann's sheath cells or myelin sheaths surrounding the nerve fibers. As a result, the conduction of neural stimuli is damaged. This leads during the further course of the disease to the increasing inability of the sensory as well as the motor nerves to maintain their function so that the disorders of sensation that are typical for multiple sclerosis as well as spastic paresis, swallowing disorders and paralysis of the musculature are produced. In many cases (ca. 80%) the primary form of the disease involves relapses with the symptoms frequently regressing to some extent after the relapse has been weathered. However, the course of the disease is usually chronic-progressive frequently with a fatal pathogenesis.

Multiple sclerosis (MS) occurs in different forms, e.g., which either can be differentiated according to its demyelination pattern of lesions in the central nervous system or lesions of the brain, which, however, is only possible by means of a biopsy, or according to its immunological mechanism, i.e., macrophage-mediated inflammation, a manifestation mediated by antibodies, a oligodentroglial part with chemical damage to the white matter (due to vasculitis of the small vessels) as well as a secondary demyelination.

Recent evidence has shown that the different forms of MS also require different therapies.

Nuclear spin resonance tomography (MRT) has also shown that the damage to the axons on the nerve endings represents a significant step in the development of irreversible nerve damage. However, to date the individually progressing immunological reactions have not been completely understood as to their interrelationship. Until now the diagnosis took place via neurological tests, e.g., through delayed nerve conduction, investigations of the liquor cerebrolumbalis by means of a lumbar puncture or the previously mentioned magnetic resonance tomography, which can visualize the inflammations and the scar tissue in the brain. A laboratory chemical investigation to date has been done by a determination of the anti-MOG antibodies (myelin oligodentrocyte glycoprotein) and anti-MBP antibodies (a corresponding protein found on the inner side of the membrane of the myelin cells (Schwann' s sheath cells)). (T. Berger in “Antimyelin antibodies as a predictor of clinically definite multiple sclerosis in patients with an initial manifestation of MS”, “New Horizons” (4), 2003, pp. 6-7).

Previously, in order to have a confirmed diagnosis of MS it was necessary to either diagnose two separate relapse phases of the disease and/or to find evidence of inflammatory foci in two different regions of the brain or spinal cord. At the same time, magnetic resonance tomography (MRT) has particularly proven its worth. With this procedure it is now also possible to show lesions to a resolution and size of 1 mm.

A test with which multiple sclerosis could be detected early on or at the beginning of a deteriorating relapse so that appropriate therapeutic measures could be initiated has not been available to date. Furthermore, a need for monitoring exists, with which the effectiveness of the therapeutic measures can be checked.

Therefore the goal of the invention is to provide such a method. This goal will be achieved through the method defined in the claims as well as the test kit that is used in the process.

According to the present invention, it was found that multiple sclerosis is accompanied by an increase in antibodies that are directed against spectrin. These antibodies can be demonstrated in bodily fluids.

Spectrins are proteins that bind to F-actin and thus promote the gelification of actin. It is found in the cytoskeleton of the cell and stabilizes the cell shape. Spectrin itself occurs as a heterotetrameric protein that consists of an alpha-spectrin 260 kDa in size and a beta-spectrin—225 kDa in size (alpha₂ beta₂). A subform, also described as a protein similar to spectrin, is fodrin. Fodrin links adjacent actin filaments with one another. In a preferred embodiment according to the invention, antibodies to fodrin are determined.

Fodrin itself has been known for some time as a differentiation marker for gastrointestinal neoplasms (M. Younes et al., Am. J. Pathol, Vol. 135 (1989),1197-1212.

In a particularly preferred embodiment according to the invention, antibodies are determined that are directed against alpha-fodrin, with antibodies directed against a neoantigen of alpha-fodrin 100 to 140 kDa or 110 to 130 kDa in size, in particular ca. 120 kDa in size, being especially preferred. Such a neoantigen, for example, is split off by a protease, particularly a caspase, during apoptosis. Typical caspases cleave peptide bonds at the C-terminal of aspartate, which is why this is also called C-Asp-ase. The splitting off of the 120 kD fragments of alpha-fodrin occurs in the body by means of caspase-3. Although autoantibodies against spectrins, in particular fodrin, can be found in all forms of MS, they are especially characteristic of subtypes 3 and 2.

In principle, the occurrence of IgA and IgG autoantibodies against alpha-fodrin is known in lupus erythematosis as well as in Sjögren syndrome. Thus WO 01/14877, for example, describes a test kit for the determination of autoantibodies against alpha-fodrin as a specific marker for the diagnosis of Sjögren syndrome. The invention relates therefore also to the use of such a test for the diagnosis and monitoring of MS as well as for the determination of subtypes 3 (primarily apoptosis) and in particular 2 (antibody-mediated).

The occurrence of significant quantities of IgA autoantibodies against alpha-fodrin is thus a typical method of demonstrating the presence of MS, especially subtypes 3 as well as 2.

At the same time, the sensitivity of the tests can be increased by the additional determination of IgA and IgG as well as IgM against the aforementioned spectrins, in particular alpha-fodrin. The additional determination of IgG autoantibodies alone increases the sensitivity by ca. 10-20%.

The inventive method can be used not only for the qualitative but also for the quantitative determination of the antibodies that occur. A quantitative determination of the autoantibodies indicates the acute status of the disease or the severity of its progression. Furthermore, relapses that respond well can also be ascertained at the start, making early and thus more successful treatment of this serious disease possible. As a result, the occurrence of irreversible damage can be minimized, possibly even prevented entirely. In principle, during a qualitative determination an antibody concentration that lies above a certain limit value is defined as a positive result. The determination of such limit values is done by calibrating the test system with sera from healthy as well as from ill patients.

According to the invention, the antibodies are usually determined in a bodily fluid. Typical bodily fluids here are blood, serum and plasma, with liquor cerebrospinalis being preferred.

The determination of the IgA or IgG antibodies to alpha-fodrin or the neoantigen ca. 120 kDa in size can be done by means of techniques known to the professional, such as an ELISA or RIA.

According to the invention, it is also possible to determine the antibody concentration by means of biosensors, e.g., amperometric sensors, potentiometric, ion-selective potentiometric or photometric sensors or also those using semi-conductor electrodes such as field-effect transistors (FET), chemosensitive field-effect transistors (CHEMFET), suspended gate field-effect transistors (SGFET) or ion-sensitive field-effect transistors. Biosensors of the kind are described comprehensively by E. A. H. Hall and G. Hummel in “Biosensors”, Springer Verlag, Heidelberg, Germany, 1995. Additional developments of ion-sensitive field-effect transistors (ISFET) or optical detectors are described by F. Aberl and H. Wolf in “Aktuelle Trends in der Immunsensorik”, Labor 2000, pp.70-96 (1993) and others. The inventive method of doing the determination by means of piezoelectrical quartz resonators and surface wave elements is also suitable; these then can be used as microscales. In the process, the primary antibody (the so-called ‘catcher’) is immobilized on a piezoelectrical substrate and measured, after binding to the GFAP to be analyzed. Such sensors are described, for example, by A. Leidl et al. in “Proceedings of the Second International Symposium on Miniaturized Total Analysis Systems μTAS”, Basel, 1996. Quartz crystal microscales, as described by C. Koslinger et al., Fresenius J. Anal. Chem. (1994), 349: 349-354, have proven especially suitable.

The alpha-fodrin antigen may be a native protein, obtained from human cell lines, or a recombinant antigen that is produced in a heterologous host cell, e.g., a bacterial cell such as e. coli or a eucaryotic host cell, such as an insect cell produced through recombinant protein expression. A recombinant alpha-fodrin antigen that contains the sequence of the native alpha-fodrin or sections there, in particular the N-terminal section, is preferred for use. The recombinant antigen can furthermore comprise heterologous peptide or polypeptide domains, e.g., a poly-His sequence which facilitates purification after expression.

The IgA-specific receptor is generally an antibody that is able to detect selectively immunoglobulins of class A in the presence of immunoglobulins of other classes, e.g., G and/or M. Polyclonal anti-IgA antisera, which can be obtained by immunizing test animals, e.g., goats, rats, mice, rabbits, etc., may be used for this purpose.

Corresponding monoclonal anti-lgA antibodies can also be used.

As already mentioned, the specific test format in general is not critical. However, the use of a heterogeneous test format, in which an immune complex consisting of spectrin, alpha-fodrin antigen in particular, IgA autoantibodies to be detected and IgA-specific receptors are bound to a solid phase (sandwich test format). However, a competitive test format can also be selected. With a heterogeneous sandwich test format one can use
(a) a spectrin immobilized on a solid phase, such as alpha-fodrin antigen, and a labeled IgA-specific receptor or
(b) an IgA-specific receptor immobilized on the solid phase and a labeled spectrin, such as alpha-fodrin antigen.

Reaction vessels, microtiter plates, beads, biochips etc. may used as solid phases. The immobilization of the antigens or the receptors on a solid phase can be achieved by adsorptive interactions, covalent bonding or mediated by a high-affinity binding pair (streptavidin/biotin, hapten/anti-hapten antibody). The immobilized test reagent can be used in a form already bound to a solid phase or else immobilized during the course of the test.

The method can be carried out as a liquid test (e.g. in a reaction vessel) or also as a dry test (e.g., on a test strip).

The labeled test reagent can itself have a detectable or signal-emitting group (direct labeling) or be capable of binding to a detectable group (indirect labeling). The labeling group can be selected at will from all labeling groups known from prior art for immunological detection methods, for example, from enzymes, metal or latex particles, and luminescent or fluorescent groups. It is particularly preferable for the labeling group to be selected from enzymes, e.g., peroxidase, β-galactosidase or alkaline phosphatase, and for the method to be carried out in the ELISA format.

The invention also relates to the use of a test kit for the diagnosis of multiple sclerosis comprising
(a) a spectrin antigen, in particular an alpha-fodrin antigen, and
(b) an IgA-specific receptor.

The test kit may additionally comprise (c) a solid phase onto which one of the test reagents (a) or (b) is bound or is capable of being bound. The test kit moreover preferably comprises (d) a labeling group that is bound to one of the test reagents (a) or (b) or to which it is capable of being bound.

In addition, the test kit may comprise (e) at least one other antibody class-specific test reagent if, besides IgA autoantibodies, a-fodrin antibodies of other immunoglobulin classes are also to be determined. Examples of such antibody class-specific test reagents are anti-IgG antibodies or protein G for selective binding of IgG autoantibodies, or anti-IgM antibodies for selective binding of IgM autoantibodies. The test kit may furthermore comprise other conventional reagents such as buffers, substrates and wetting solutions.

The invention should be further illustrated by the following examples:

### EXAMPLE

Sera from 99 patients suffering from clinically stable MS as well as 27 patients who have had an acute relapse were investigated and treated with high doses of intravenously administered steroids. The serum was taken before and after treatment. Not only untreated patients but also patients who had been treated with interferon B or glatiramer acetate were accepted into the study. The sera were frozen at minus 20° C. and later investigated for the presence of IgG and IgA alpha-fodrin antibodies by means of a conventional ELlSA (AESKULISA alpha-fodrin A from Aesku. Diagnostics, Wendelsheim, Del.) as well as for antinuclear antibodies (ANA) from Hep2-cells (Aesku. slides from Aesku. Diagnostics, Wendelsheim, Del.). At the same time, the concentration of these antibodies was also determined and the sera that exceeded the limit value of 15 U/ml declared positive, and in fact not only for the IgG but also for the IgA values.

6 out of the 99 patients (6.06%) showed an elevated IgG and/or IgA anti-alpha-fodrin level compared with 10 out of the 27 patients (37.04%) with acute relapses (P<0.001). The antibody level decreased in all patients after treatment with steroids. In 26 clinically stable patients serum was taken repeatedly over a period of one year (after 0, 1, 3, 6 and 12 months). It appeared that the antibody level in these patients remained the same. In 77 patients with MS the test for ANA showed a value greater than or equal to 1:320 (23.4%) and in 49.4% of the patients a value greater than or equal to 1:160. A connection between the anti-alpha-fodrin titers and the ANA could not be ascertained. Alpha-fodrin or its neoantigen ca. 120 kDa in size is thus a significant indicator for the occurrence of MS relapses, particularly for subtype 3 and especially for subtype 2.

| | IgA-Fodrin | IgG-Fodrin |
|---|---|---|
| Patient | (μ/ml) | (μ/ml) |
| 1 | 3 | 24 |
| 2 | 2 | 36 |
| 3 | 30 | 8 |
| 4 | 7 | 13 |
| 5 | 3 | 19 |
| 6 | 27 | 47 |
| 7 | 5 | 13 |
| 8 | 19 | 14 |
| 9 | 48 | 28 |
| 10 | 4 | 3 |
| 11 | 3.2 | 3.9 |
| 12 | 6.5 | 11.6 |
| 13 | 3.1 | 4.8 |
| 14 | 10.7 | 6.2 |
| 15 | 29.5 | 6.2 |
| 16 | 5.8 | 8.8 |
| 17 | 47.6 | 32.1 |
| 18 | 8.1 | 6.1 |
| 19 | 10.1 | 11.1 |
| 20 | 11 | 5.2 |
| 21 | 1 | 3.4 |
| 22 | 26.7 | 27.9 |
| 23 | 19.8 | 4.6 |
| 24 | 4.6 | 4.3 |
| 25 | 6.4 | 4 |
| 26 | 6.5 | 5.1 |
| 27 | 14.2 | 6.3 |
| 28 | 11.1 | 6.1 |
| 29 | 6.9 | 6.8 |
| 30 | 15.1 | 6.9 |
| 31 | 3.2 | 3.8 |

1. A method of diagnosing and monitoring multiple sclerosis by determining autoantibodies in bodily fluid, comprising:
measuring the amount of spectrin-binding antibodies in bodily fluid; and
determining whether the amount of spectrin-binding antibodies in said bodily fluid is higher than the amount of spectrin-binding antibodies in the same type of bodily fluid from a healthy patient.

2. The method of claim 1, wherein the spectrin is fodrin.

3. The method of claim 2, wherein the antibodies are directed against the alpha form of fodrin.

4. The method of claim 1, wherein the antibody is directed against a spectrin fragment 100 to 140 kDa in size.

5. The method of claim 1, wherein the antibodies are directed against a neoantigen of fodrin 120 kDa in size, which is cleavable by caspase-3.

6. The method of claim 1, wherein the autoantibodies that are measured are IgA and IgG autoantibodies.

7. The method of claim 1, wherein the antibodies are directed against a member of the group consisting of the alpha form of fodrin and a neoantigen of fodrin 120 kDa in size and cleavable by caspase-3, wherein an elevated level of antibodies is indicative of at least one subtype of multiple sclerosis selected from the group consisting of subtype 2 and subtype 3.

8. A test kit for diagnosing and monitoring multiple sclerosis comprising test reagents (a) a spectrin antigen and (b) a specific receptor for an autoantibody, wherein said autoantibody binds to spectrin.

9. The test kit of claim 8, wherein the spectrin antigen is an alpha-fodrin antigen and the receptor is an IgA- or IgG-specific receptor.

10. The test kit of claim 9, comprising a solid phase onto which one of the test reagents (a) or (b) is bound or is capable of binding.

11. The test kit of claim 10, further comprising a labeling group, said labeling group bound to or capable of binding to one of the test reagents selected from the group consisting of test reagent (a) and test reagent (b).

12. The test kit of claim 9, wherein the kit comprises at least one additional antibody class-specific test reagent.

13. The test kit of claim 9, further comprising instructions for using the test kit for detection of multiple sclerosis.
